(19) United States
(12) Patent Application Publication
(10) Pub. No.: US 2022/0031285 A1
(43) Pub. Date: Feb. 3, 2022

(54) ULTRASONIC DIAGNOSTIC APPARATUS AND STORAGE MEDIUM

(71) Applicant: CANON MEDICAL SYSTEMS CORPORATION, Otawara-shi (JP)

(72) Inventors: Yuya NAGASAKI, Nasushiobara (JP); Yusuke KOBAYASHI, Nasushiobara (JP); Kentaro TSUZUKI, Nasushiobara (JP)

(73) Assignee: CANON MEDICAL SYSTEMS CORPORATION, Otawara-shi (JP)

(21) Appl. No.: 17/305,603

(22) Filed: Jul. 12, 2021

(30) Foreign Application Priority Data
Jul. 29, 2020 (JP) 2020-128074

(51) Int. Cl. A61B 8/00; A61B 8/14; A61B 8/02; A61B 8/08

(52) U.S. Cl. CPC A61B 8/463; A61B 8/14; A61B 8/5253; A61B 8/02; A61B 8/4263

(57) ABSTRACT

An ultrasonic diagnostic apparatus of an embodiment includes processing circuitry. The processing circuitry is configured to acquire an image of a subject based on a reflected echo signal obtained from reflection of an ultrasonic signal, which is transmitted from an ultrasonic probe, from the subject, identify an acquisition position in the subject at which the image has been acquired, identify a time phase in which the image has been acquired, and cause a display to display an acquisition range of the image on the subject in at least one specific time phase area based on the identified acquisition position and time phase.

### CROSS-REFERENCE TO RELATED APPLICATION

The present application claims priority based on Japanese Patent Application No. 2020-128074 filed Jul. 29, 2020, the content of which is incorporated herein by reference.

### FIELD

Embodiments disclosed in the present description and drawings relate to an ultrasonic diagnostic apparatus and a storage medium.

### BACKGROUND

When the size of an image obtained using an ultrasonic probe with respect to an area that is an examination target is small, for example, a plurality of images are captured while the ultrasonic probe is operated and an image of the area that is the examination target is generated according to image compositing. In this case, images necessary to generate a composite image need to be collected without omission. Accordingly, there are techniques of performing scanning according to mechanical control or hiding a movement trajectory of an ultrasonic probe on a body mark.

However, when images are captured, it is difficult to confirm whether an image associated with a specific time phase necessary for examination has been obtained because images are not connected with time phase information, and thus there are cases in which images are incompletely collected or recollection of images due to incomplete collection of images is required.

### BRIEF DESCRIPTION OF THE DRAWINGS

FIG. 1 is a block diagram showing a functional configuration of an ultrasonic diagnostic system 1 of a first embodiment.

FIG. 2 is a diagram showing an example of an image R14 displayed by a display 14 of the first embodiment.

FIG. 3 is a flowchart showing an example of processing of an ultrasonic diagnostic apparatus 100 of the first embodiment.

FIG. 4 is a diagram showing an example of an image R14 displayed by a display 14 of a second embodiment.

FIG. 5 is a diagram showing an example of an image R14 displayed by a display 14 of a third embodiment.

FIG. 6 is a diagram showing an example of an image R14 displayed by a display 14 of a fourth embodiment.

FIG. 7 is a block diagram showing a functional configuration of an ultrasonic diagnostic system 1 of a fifth embodiment.

FIG. 8 is a diagram showing an example of an image R14 displayed by a display 14 of the fifth embodiment.

FIG. 9 is a flowchart showing an example of processing of an ultrasonic diagnostic apparatus 100 of a sixth embodiment.

FIG. 10 is a block diagram showing a functional configuration of an ultrasonic diagnostic system 1 of a seventh embodiment.

FIG. 11 is a diagram showing an example of an image R14 displayed by a display 14 of the seventh embodiment.

FIG. 12 is a block diagram showing a functional configuration of an ultrasonic diagnostic system 1 of an eighth embodiment.

### DETAILED DESCRIPTION

Hereinafter, an ultrasonic diagnostic apparatus and a storage medium of embodiments will be described with reference to the drawings.

An ultrasonic diagnostic apparatus of an embodiment includes processing circuitry. The processing circuitry is configured to acquire an image of a subject based on a reflected echo signal obtained from reflection of an ultrasonic signal, which is transmitted from an ultrasonic probe, from the subject, identify an acquisition position in the subject at which the image has been acquired, identify a time phase in which the image has been acquired, and cause a display to display an acquisition range of the image on the subject in at least one specific time phase area based on the identified acquisition position and time phase.

### First Embodiment

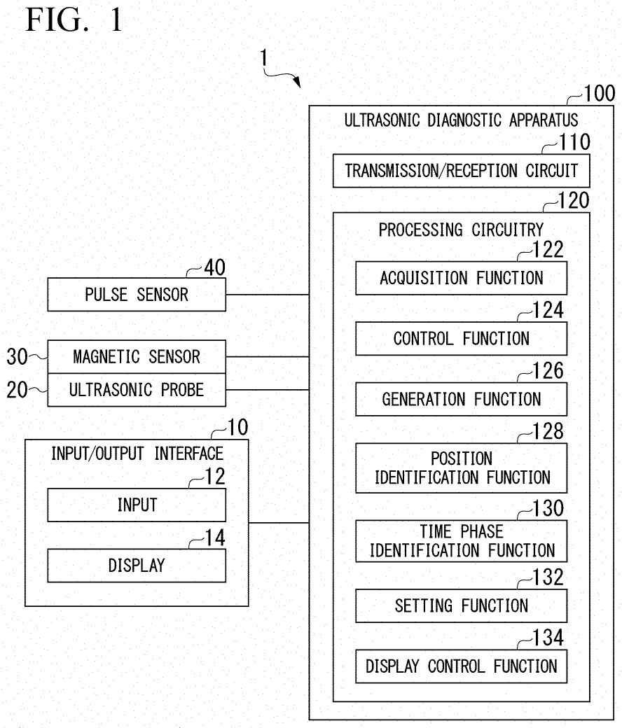

FIG. 1 is a block diagram showing a functional configuration of an ultrasonic diagnostic system 1 of a first embodiment. The ultrasonic diagnostic system 1 includes an input/output interface 10, an ultrasonic probe 20, a magnetic sensor 30, a pulse sensor 40, and an ultrasonic diagnostic apparatus 100.

The input/output interface 10 may include, for example, an input 12 and a display 14. The input 12 may include, for example, physical operation parts such as a mouse, a keyboard, and a touch panel. The input 12 generates operation information, examination target information, and time phase area information on the basis of amounts of operations of operation parts performed by an operator.

The operation information is information about control of the ultrasonic probe 20. The examination target information is information for defining a region that is a target of a subject which will be examined using the ultrasonic probe 20 (hereinafter referred to as an “examination target region”), for example, a spatial area such as “leg” or “abdomen.” The time phase area information is information indicating a range for setting a time phase area that is an examination target (hereinafter referred to as a “specific time phase area”). The specific time phase area may be, for example, a temporal area such as “diastole,” “initial stage of diastole,” or “transition from diastole to systole” in repetition of vascular dilations and contractions. The input 12 outputs the generated operation information, examination target information, and time phase area information to the ultrasonic diagnostic apparatus 100.

The input 12 is not limited to a component including physical operation parts such as a mouse and a keyboard. The input 12 may be, for example, electrical signal-processing circuitry that receives an electrical signal associated with an input operation from an external input device provided separately from the device and outputs this electrical signal to a control circuit.

The display 14 may be disposed, for example, at a position at which it may display images visually recognizable by an operator such as a doctor or an engineer. The display 14 displays an image based on information output from the ultrasonic diagnostic apparatus 100. The display 14 may be, for example, a display or a projector that projects an image. When the input 12 is a touch panel, the touch panel also serves as the display 14.

The ultrasonic probe 20 transmits an ultrasonic signal to a subject. The ultrasonic probe 20 receives a reflected echo signal reflected from the subject. The ultrasonic probe 20 generates reflected wave information on the basis of the received reflected echo signal. The ultrasonic probe 20 outputs the generated reflected wave information to the ultrasonic diagnostic apparatus 100.

The magnetic sensor 30 may be laid on the ultrasonic probe 20, for example. The magnetic sensor 30 may be embedded in the ultrasonic probe 20. The magnetic sensor 30 detects a position of the ultrasonic probe 20, for example, on the basis of change in magnetic fields. The magnetic sensor 30 outputs the detected position of the ultrasonic probe 20 to the ultrasonic diagnostic apparatus 100 as position information.

The pulse sensor 40 is attached to the ultrasonic diagnostic apparatus 100. The pulse sensor 40 acquires pulse information as biometric information of a subject. The pulse sensor 40 is attached to the body of a subject, for example, a wrist, at the time of examination of the subject. The pulse sensor 40 attached to the body of the subject detects a pulse of the subject. The pulse sensor 40 outputs pulse information based on the detected pulse to the ultrasonic diagnostic apparatus 100.

The ultrasonic diagnostic apparatus 100 may include, for example, a transmission/reception circuit 110 and processing circuitry 120. The transmission/reception circuit 110 transmits/receives various types of information to/from devices such as the input/output interface 10 and the ultrasonic probe 20. Further, the transmission/reception circuit 110 may apply, for example, a transmission voltage to the ultrasonic probe 20.

The transmission/reception circuit 110 receives reflected wave information output from the ultrasonic probe 20. The transmission/reception circuit 110 converts the received reflected wave information into a digital signal. The transmission/reception circuit 110 outputs the reflected wave information converted into the digital signal to the processing circuitry 120. The transmission/reception circuit 110 receives operation information, examination target information, and time phase area information output from the input 12.

The transmission/reception circuit 110 receives position information output from the magnetic sensor 30. The transmission/reception circuit 110 receives pulse information output from the pulse sensor 40. The transmission/reception circuit 110 outputs the received position information, pulse information, operation information, examination target information, and time phase area information to the processing circuitry 120.

The processing circuitry 120 may include, for example, an acquisition function 122, a control function 124, a generation function 126, a position identification function 128, a time phase identification function 130, a setting function 132, and a display control function 134. The processing circuitry 120 realizes these functions, for example, by a hardware processor executing programs stored in a storage device (storage circuit).

The hardware processor may refer to, for example, circuitry such as a central processing unit (CPU), a graphics-processing unit (GPU), an application-specific integrated circuit (ASIC), or a programmable logic device (e.g., a simple programmable logic device (SPLD), a complex programmable logic device (CPLD), or a field-programmable gate array (FPGA)). The programs may be directly incorporated in the circuit of the hardware processor instead of being stored in the storage device. In this case, the hardware processor realizes the functions by reading and executing the programs incorporated in the circuit thereof. The hardware processor is not limited to a configuration of a single circuit, and a plurality of independent circuits may be combined as a single hardware processor to realize each function. In addition, a plurality of components may be integrated into a single hardware processor to realize each function. The programs may be stored in a non-transitory (hardware) storage medium.

The acquisition function 122 acquires reflected wave information, operation information, examination target information, time phase area information, position information, and pulse information output from the transmission/reception circuit 110. The acquisition function 122 outputs the acquired reflected wave information to the generation function 126. The acquisition function 122 outputs the acquired operation information to the control function 124. The acquisition function 122 outputs the acquired examination target information and the time phase area information to the setting function 132. The acquisition function 122 outputs the acquired position information to the position identification function 128. The acquisition function 122 outputs the acquired pulse information to the time phase identification function 130 and the display control function 134. The acquisition function 122 is an example of an acquirer.

The control function 124 identifies the transmission voltage applied to the ultrasonic probe 20 on the basis of the operation information output from the input 12. The control function 124 generates transmission/reception conditions according to the identified transmission voltage. The control function 124 applies the transmission voltage according to the generated transmission/reception conditions to a pulser of the transmission/reception circuit 110 with respect to the ultrasonic probe 20.

The generation function 126 generates an ultrasonic image that is an image of the inside of the subject on the basis of the reflected wave information output from the ultrasonic probe 20. The generation function 126 outputs the generated ultrasonic image to the acquisition function 122. The acquisition function 122 outputs the acquired ultrasonic image to the display control function 134. The acquisition function 122 is an example of an acquirer. The generation function 126 serves as the acquirer. The control function 124 and the generation function 126 may be provided outside the processing circuitry 120. When the generation function 126 is provided outside the processing circuitry 120, the generation function 126 outputs the generated ultrasonic image to the acquisition function 122. The generation function 126 may output the ultrasonic image to the display control function 134 instead of the acquisition function 122.

The position identification function 128 identifies an acquisition position that is a position of the ultrasonic probe 20 and a spatial position in the subject at which the ultrasonic probe 20 has acquired the ultrasonic image on the basis of the position information output from the magnetic sensor 30. The position identification function 128 outputs acquisition position information indicating the identified acquisition position to the display control function 134. The position identification function 128 is an example of a position identifier.

The time phase identification function 130 identifies a time phase as temporal information, in which the ultrasonic probe 20 has received the reflected echo signal for generating the ultrasonic image, on the basis of the pulse information output from the pulse sensor 40. The time phase identification function 130 generates time phase information indicating the identified time phase and outputs the time phase information to the display control function 134. The time phase identification function 130 may identify the time phase, for example, on the basis of periodically or aperiodically varying biometric information of the subject other than the pulse information, for example, heart rate information or respiration information. The time phase identification function 130 is an example of a time phase identifier.

The setting function 132 synchronizes position information using the position information output from the ultrasonic probe 20. The setting function 132 sets an acquisition range of an examination target region in the subject on the basis of the examination target information output from the input 12. The setting function 132 may present a plurality of acquisition ranges to the operator such that the operator selects an acquisition range to be set from the plurality of acquisition ranges.

The setting function 132 generates time variation information indicating time variation in the pulse information that is biometric information on the basis of the pulse information output from the pulse sensor 40 and outputs the time variation information to the display control function 134. The display control function 134 visualizes the output time variation information and causes the display 14 to display the visualized time variation information. The time variation information may be represented as, for example, a waveform in a coordinate space having an opening degree of a blood vessel as a vertical axis and time as a horizontal axis.

The setting function 132 causes the operator to set a specific time phase area by causing the operator to designate a part of the time variation information displayed on the display 14. A time length of the specific time phase area may be adjusted by the operator. The input 12 is configured such that the operator can designate a part of the time variation information displayed on the display 14 through the input 12. The input 12 generates time phase area information on the basis of a range of time variation information designated by the operator.

The setting function 132 sets a specific time phase area for identifying an acquisition range of the examination target region on the basis of the time phase area information output from the input 12. The setting function 132 sets one or a plurality of specific time phase areas. In the first embodiment, the setting function 132 sets two specific time phase areas. The setting function 132 outputs setting information based on the set acquisition range of the examination target region and specific time phase area to the display control function 134. The specific time phase area may be, for example, an area set in advance instead of being set by the setting function 132, and in this case, may be set according to the examination target region or may be set according to an examination item, for example. The setting function 132 may present a plurality of specific time phase areas to the operator such that the operator selects a specific time phase area to be set from the plurality of specific time phase areas. The setting function 132 is an example of a setter.

The display control function 134 causes the display 14 to display the ultrasonic image output from the acquisition function 122. The display control function 134 generates an image representing a body mark on the basis of the examination target information output from the input 12 and causes the display 14 to display the body mark. The body mark may be, for example, information representing the examination target region as a mimic image. For example, when the examination target region is a leg, the display control function 134 causes the display 14 to display an image imitating the leg.

The display control function 134 labels an acquisition position based on the acquisition position information output from the position identification function 128 and a time phase based on the time phase information output from the time phase identification function 130 on the ultrasonic image output from the acquisition function 122. The display control function 134 determines whether the time phase labeled on the ultrasonic image (hereinafter referred to as a “labeled time phase”) is included in the specific time phase area on the basis of the setting information output from the setting function 132.

When the labeled time phase is included in the specific time phase area, the display control function 134 identifies an acquisition position in the subject at which the ultrasonic image has been acquired on the basis of the acquisition position labeled on the ultrasonic image (hereinafter referred to as a “labeled acquisition position”). After identification of the acquisition position, the display control function 134 identifies an acquisition range of the ultrasonic image on the basis of the identified acquisition position. The display control function 134 causes the display 14 to display the identified acquisition range. The display control function 134 causes the identified acquisition range to be superimposed and displayed on the body mark. The display control function 134 is an example of a display controller.

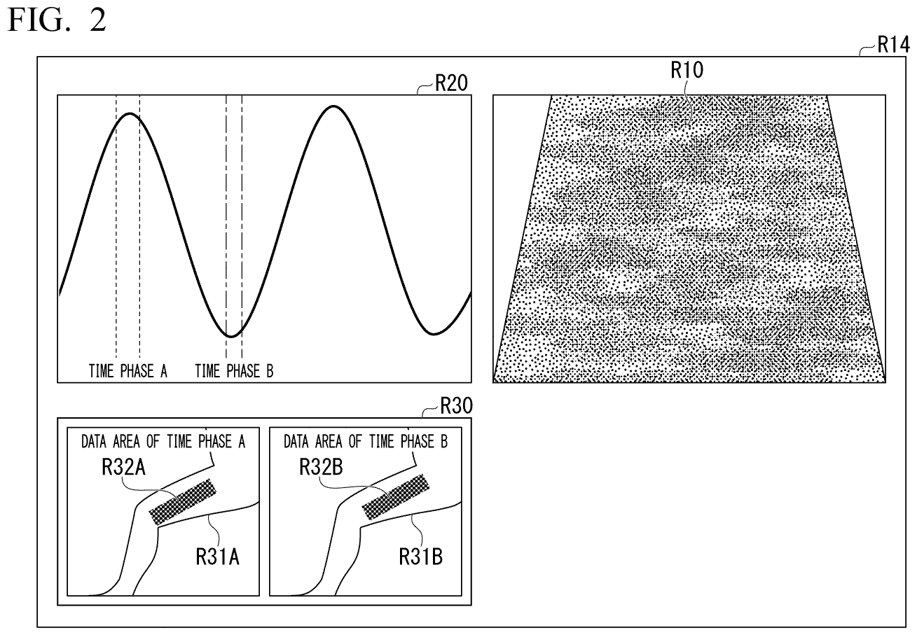

FIG. 2 is a diagram showing an example of an image R14 displayed by the display 14 of the first embodiment. Here, the display control function 134 causes the display 14 to display two specific time phase areas of “time phase A” and “time phase B.” “Time phase A” is a time phase corresponding to “diastole” and “time phase B” is a time phase corresponding to “systole.” A time length of “time phase A” is set to be longer than a time length of “time phase B.”

An ultrasonic image R10, a time variation image R20, and an acquisition range display image R30 are displayed on the display 14. In a waveform representing the time variation image R20, an upwardly convex part is “diastole” in which blood vessels dilate to the maximum and a downwardly convex part is “systole” in which the blood vessels constrict to the maximum.

The acquisition range display image R30 includes a first body mark image R31A and a second body mark image R31B in the first embodiment. A first acquisition range image R32A is superimposed and displayed on the first body mark image R31A and a second acquisition range image R32B is superimposed and displayed on the second body mark image R31B. The first body mark image R31A and the second body mark image R31B have the same shape. The first acquisition range image R32A represents a range in which acquisition positions of ultrasonic images having a labeled time phase of time phase A (diastole) are collected. The second acquisition range image R32B represents a range in which acquisition positions of ultrasonic images having a labeled time phase of time phase B (systole) are collected.

The first acquisition range image R32A is an image generated on the basis of a labeled acquisition position of an ultrasonic image having a labeled time phase of time phase A (diastole). The second acquisition range image R32B is an image generated on the basis of a labeled acquisition position of an ultrasonic image having a labeled time phase of time phase B (systole). In the following description, the first body mark image R31A and the second body mark image R31B are represented as a body mark image R31 when they need not be particularly distinguished from each other, and the first acquisition range image R32A and the second acquisition range image R32B are represented as an acquisition range image R32 when they need not be particularly distinguished from each other.

The display control function 134 causes the display 14 to display both the first acquisition range image R32A and the second acquisition range image R32B through colored visual display. The display control function 134 causes the display 14 to display the first acquisition range image R32A and the second acquisition range image R32B having different labeled time phases side by side. The first acquisition range image R32A and the second acquisition range image R32B may be in the same color or different colors. The first acquisition range image R32A and the second acquisition range image R32B may be displayed through visual display other than coloring, for example, hatching or texture, or using a combination thereof.

Next, with respect to processing in the ultrasonic diagnostic apparatus 100, processing before initiation of ultrasonic diagnosis will be described. In initiation of ultrasonic diagnosis, the display control function 134 causes the display 14 to display a body mark image R31 associated with an acquisition range set on the basis of an operation performed on the input 12 by an operator and performs synchronization processing. As synchronization processing, the display control function 134 first causes an origin image representing an origin position to be displayed on the body mark image R31 for alignment. In this state, the operator operates the ultrasonic probe 20 to adjust an acquisition position to the origin image. Here, the operator presses, for example, a designation button that is not illustrated such that the display control function 134 synchronizes position information. The display control function 134 executes this synchronization processing at two points on the body mark image R31 to complete position synchronization including a direction. The number of points at which synchronization processing is performed varies according to the shape of the body mark image R31 and the like.

Subsequently, the operator designates a part of a period represented in a time variation image R20 to set a specific time phase area. As the time variation image R20, for example, a graph showing time variation in pulses of one period or more is drawn. The operator designates a starting point and an end point of the specific time phase area by operating the input 12 to set the specific time phase area. Two specific time phase areas are set in the first embodiment. In this manner, the operator initiates ultrasonic diagnosis after setting of the acquisition area and the specific time phase area and synchronization processing. The acquisition area and the specific time phase area may be set in advance.

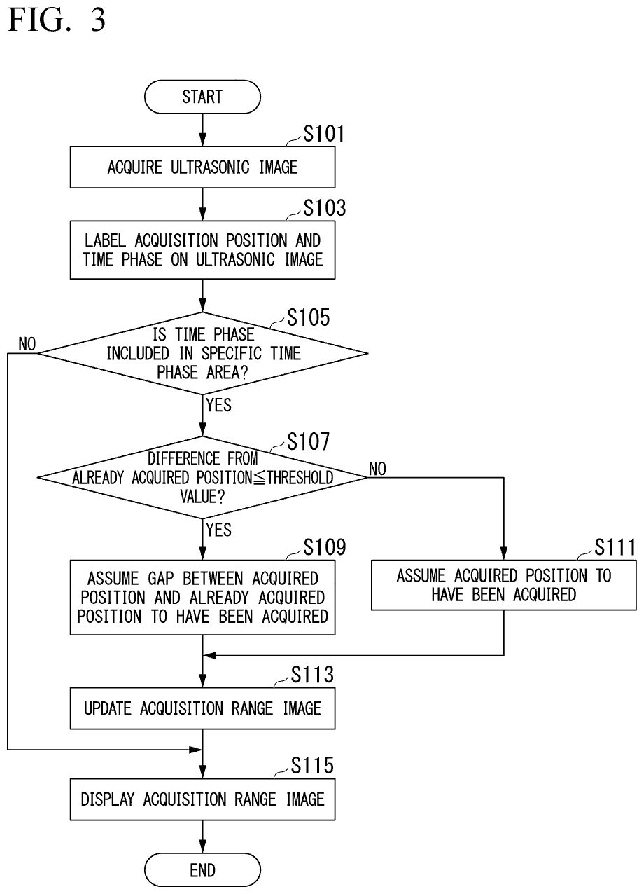

Next, processing in the ultrasonic diagnostic apparatus 100 will be described. FIG. 3 is a flowchart showing an example of processing of the ultrasonic diagnostic apparatus 100 of the first embodiment. Here, processing after completion of synchronization processing and setting of the acquisition area and the specific time phase area will be described. The ultrasonic diagnostic apparatus 100 may execute processing of the flowchart shown in FIG. 3, for example, whenever the ultrasonic probe 20 outputs reflected wave information to the ultrasonic diagnostic apparatus 100. First, the generation function 126 of the ultrasonic diagnostic apparatus 100 generates an ultrasonic image on the basis of reflected wave information output from the ultrasonic probe 20 and outputs the ultrasonic image to the acquisition function 122. The acquisition function 122 acquires the ultrasonic image output from the generation function 126 (step S101).

In acquisition of an ultrasonic image, the ultrasonic diagnostic apparatus 100 causes the ultrasonic probe 20 to transmit an ultrasonic signal and to receive a reflected echo signal. The ultrasonic diagnostic apparatus 100 generates and acquires an ultrasonic image through the generation function 126 on the basis of reflected wave information output from the ultrasonic probe 20 and acquired through the acquisition function 122. The ultrasonic diagnostic apparatus 100 generates and acquires a plurality of ultrasonic images by repeating the same processing a plurality of times.

Upon acquisition of an ultrasonic image, the position identification function 128 identifies an acquisition position and the time phase identification function 130 identifies a time phase. Subsequently, the display control function 134 labels the acquisition position identified by the position identification function 128 and the time phase identified by the time phase identification function 130 on the ultrasonic image (step S103).

Subsequently, the display control function 134 determines whether the time phase labeled on the ultrasonic image is included in a specific time phase area on the basis of setting information output from the setting function 132 (step S105). When it is determined that the time phase labeled on the ultrasonic image is not included in the specific time phase area, the ultrasonic diagnostic apparatus 100 proceeds to step S115.

When it is determined that the time phase labeled on the ultrasonic image is included in the specific time phase area, the display control function 134 calculates a positional deviation of the acquisition position labeled on the acquired ultrasonic image from an acquisition position of a closest ultrasonic image among ultrasonic images acquired in the past (hereinafter referred to as an “already acquired ultrasonic image”). The display control function 134 determines whether the calculated position deviation is equal to or less than a predetermined threshold value (step S107).

When it is determined that the calculated position deviation is equal to or less than the predetermined threshold value, the display control function 134 assumes that a gap between the acquisition position of the currently acquired ultrasonic image and the acquisition position of the already acquired ultrasonic image has been acquired (step S109). When it is determined that the calculated positional deviation is not equal to or less than the predetermined threshold value (exceeds the threshold value), the display control function 134 assumes that the acquisition position of the acquired ultrasonic image has been acquired (step S111).

Subsequently, the display control function 134 updates an acquisition range image R32 associated with the time phase labeled on the acquired ultrasonic image on the basis of the acquisition position of the currently acquired ultrasonic image (step S113). Subsequently, the display control function 134 causes the display 14 to display the updated acquisition range image R32 and a body mark image R31. In this manner, the ultrasonic diagnostic apparatus 100 ends processing shown in FIG. 3.

In the ultrasonic diagnostic apparatus 100 of the first embodiment, the display control function 134 labels a time phase on an ultrasonic image acquired by the acquisition function 122 and calculates a position at which an ultrasonic image has been acquired for each specific time phase area. The ultrasonic diagnostic apparatus 100 generates the acquisition range image R32 on the basis of the position at which the ultrasonic image has been acquired for each specific time phase area and causes the display 14 to display the acquisition range image R32 and the body mark image R31. Accordingly, the operator can easily recognize an area having insufficient ultrasonic images for each specific time phase area and thus can easily recognize a time phase having insufficient ultrasonic images. Therefore, it is possible to confirm a spatiotemporal collection state of images.

In the ultrasonic diagnostic apparatus 100 of the first embodiment, an acquisition position is identified on the basis of a detection result of the magnetic sensor 30 provided in the ultrasonic probe 20. Accordingly, it is possible to easily identify an acquisition position without providing an additional member for identifying the acquisition position.

In the ultrasonic diagnostic apparatus 100 of the first embodiment, the time phase identification function 130 identifies a time phase in which the ultrasonic probe 20 has acquired an ultrasonic image on the basis of pulse information output from the pulse sensor 40. Accordingly, it is possible to easily identify a time phase in which an ultrasonic image has been acquired.

In the ultrasonic diagnostic apparatus 100 of the first embodiment, the setting function 132 sets a specific time phase area for identifying a time phase on the basis of time phase area information output from the input 12. Accordingly, it is possible to easily set a specific time phase area.

In the ultrasonic diagnostic apparatus 100 of the first embodiment, the display control function 134 causes the display 14 to display the body mark image R31 and causes the acquisition range image R32 to be superimposed and displayed on the body mark image R31. Accordingly, it is possible to display an acquisition state of an ultrasonic image on the body mark image R31 such that the acquisition state can be easily understood.

In the ultrasonic diagnostic apparatus 100 of the first embodiment, the display control function 134 causes the display 14 to display the first acquisition range image R32A and the second acquisition range image R32B having different labeled time phases side by side. Accordingly, it is possible to cause the operator to recognize an acquisition range image such that the operator hardly confuses the first and second acquisition range images R32A and R32B.

Although a time phase is detected using the pulse sensor 40 and identified in the ultrasonic diagnostic apparatus 100 of the first embodiment, a time phase may be detected using other detection means and the like and identified. For example, a heart rate sensor that detects a heart rate of a subject may be used. Otherwise, a cardiac cycle of a subject may be measured or measured and stored in advance, a start point at which ultrasonic diagnosis is initiated is measured, and then a time phase may be identified using the cardiac cycle of the subject. By using these devices, a time phase in which an ultrasonic image is acquired can be identified in various forms.

Although an acquisition range image is superimposed and displayed on the body mark image R31 in the first embodiment, the acquisition range image may be displayed on images other than the body mark image R31, and a reference image of the subject may be captured and the acquisition range image may be superimposed and displayed on the captured reference image, for example. The reference image may be, for example, a computed tomography (CT) image, a magnetic resonance (MR) image, an ultrasonography (UL) image, or a picture image. The acquisition range image may be displayed without being superimposed on the body mark image R31 and the like and the acquisition range image may be displayed without displaying the body mark image R31 and the like. By using the reference image and the like, the body mark image R31 need not be generated.

The display control function 134 may cause the display 14 to enlarge and display an acquisition range image according to an operation performed on the input 12 by the operator. In a case where a plurality of acquisition range images R32 are displayed on the display 14, when an operation for designating one of the plurality of acquisition range images R32 is performed on the input 12 by the operator, the display control function 134 may cause the designated image to be enlarged and displayed.

Although two specific time phase areas are set in the first embodiment, only one specific time phase area may be set or two or more specific time phase areas may be set. When two or more specific time phase areas are set, the display control function 134 may cause the display 14 to display acquisition ranges for all the specific time phase areas or cause the display 14 to display acquisition ranges for some specific time phase areas.

### Second Embodiment

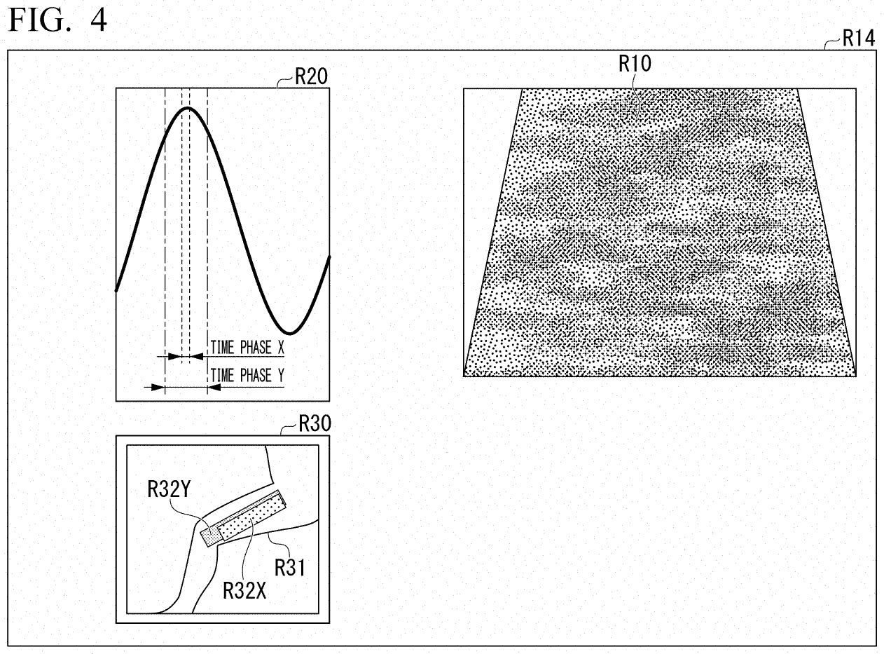

Next, an ultrasonic diagnostic apparatus 100 of a second embodiment will be described. The ultrasonic diagnostic apparatus 100 of the second embodiment mainly differs from the first embodiment with respect to images caused to be displayed on the display 14 by the display control function 134. Hereinafter, the second embodiment will be described focusing on differences from the first embodiment. FIG. 4 is a diagram showing an example of an image R14 displayed by the display 14 of the second embodiment. In FIG. 4, a time variation image R20 and an acquisition range display image R30 displayed on the display 14 are represented and display of an ultrasonic image R10 is omitted.

In the ultrasonic diagnostic apparatus 100 of the second embodiment, the display control function 134 shown in FIG. 1 causes the display 14 to display the time variation image R20 and the acquisition range display image R30 as in the first embodiment. In the ultrasonic diagnostic apparatus 100 of the second embodiment, the display control function 134 causes the display 14 to display the time variation image R20 including two specific time phase areas of “time phase X” and “time phase Y” here. “Time phase X” is shorter than “time phase Y” and “time phase Y” includes “time phase X.”

In the ultrasonic diagnostic apparatus 100 of the second embodiment, the acquisition range display image R30 includes a body mark image R31, a first acquisition range image R32X, and a second acquisition range image R32Y. The first acquisition range image R32A and the second acquisition range image R32B are independent and respectively superimposed and displayed on the first body mark image R31A and the second body mark image R31B in the first embodiment, whereas the first acquisition range image R32X and the second acquisition range image R32Y are superimposed and displayed in the second embodiment. When a plurality of acquisition range images R32 are superimposed and displayed, the display control function 134 may cause all the acquisition range images R32 to be superimposed and displayed on the body mark image R31 or cause acquisition range images R32 for some specific time phase areas to be superimposed and displayed on the body mark image R31.

The first acquisition range image R32X is an image generated on the basis of a labeled acquisition position of an ultrasonic image having a labeled time phase of “time phase X.” The second acquisition range image R32Y is an image generated on the basis of a labeled acquisition position of an ultrasonic image having a labeled time phase of “time phase Y.”

The display control function 134 displays the first acquisition range image R32X in red, for example, and displays the second acquisition range image R32Y in blue. In this manner, the display control function 134 causes the first acquisition range image R32X and the second acquisition range image R32Y to be superimposed and displayed through different visual display manners.

The ultrasonic diagnostic apparatus 100 of the second embodiment achieves the same effects as those of the ultrasonic diagnostic apparatus 100 of the first embodiment. In the ultrasonic diagnostic apparatus 100 of the second embodiment, the first acquisition range image R32X and the second acquisition range image R32Y are superimposed and displayed in the acquisition range display image R30. Accordingly, a tester can confirm the first acquisition range image R32X and the second acquisition range image R32Y in a state in which they can be compared and restrict a space in which the acquisition range display image R30 is displayed.

### Third Embodiment

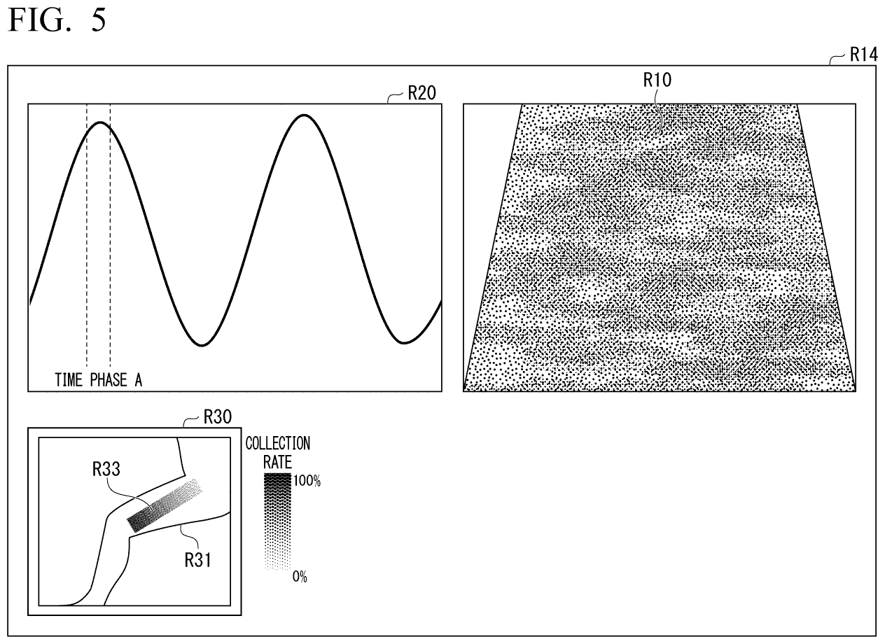

Next, an ultrasonic diagnostic apparatus 100 of a third embodiment will be described. The ultrasonic diagnostic apparatus 100 of the third embodiment mainly differs from the first embodiment with respect to images caused to be displayed on the display 14 by the display control function 134. Hereinafter, the third embodiment will be described focusing on differences from the first embodiment. FIG. 5 is a diagram showing an example of an image R14 displayed by the display 14 in the ultrasonic diagnostic apparatus 100 of the third embodiment. In FIG. 5, the acquisition range display image R30 displayed on the display 14 is represented and display of the ultrasonic image R10 and the time variation image R20 is omitted.

In the ultrasonic diagnostic apparatus 100 of the third embodiment, the display control function 134 calculates a collection rate of ultrasonic images at a position in an acquisition range image. The display control function 134 adjusts a display form of the acquisition range image R33 according to a collection rate of ultrasonic images at each position in the acquisition range image. A collection rate refers to, for example, a proportion of an area in which ultrasonic images have been acquired to a unit area. The acquisition range display image R30 includes a body mark image R31 and an acquisition range image R33. The acquisition range image R33 may be, for example, an image in shades varying according to display position.

In the acquisition range image R33, a collection rate of ultrasonic images in a part displayed in dark is high and a collection rate of ultrasonic images in a part displayed in light is low, as shown in FIG. 5. The acquisition range image R33 is displayed in gradation. An acquisition range image may be divided and displayed for collection rates. In FIG. 5, a collection rate of ultrasonic images is high in a bottom left part in the acquisition range represented in the acquisition range image R33 and the collection rate of ultrasonic images becomes lower towards the top right part.

The ultrasonic diagnostic apparatus 100 of the third embodiment achieves the same effects as those of the ultrasonic diagnostic apparatus 100 of the first embodiment. The ultrasonic diagnostic apparatus 100 of the third embodiment adjusts a display form of the acquisition range image R33 according to a collection rate of ultrasonic images. Accordingly, an operator can easily ascertain a position at which a collection rate of ultrasonic images is low.

### Fourth Embodiment

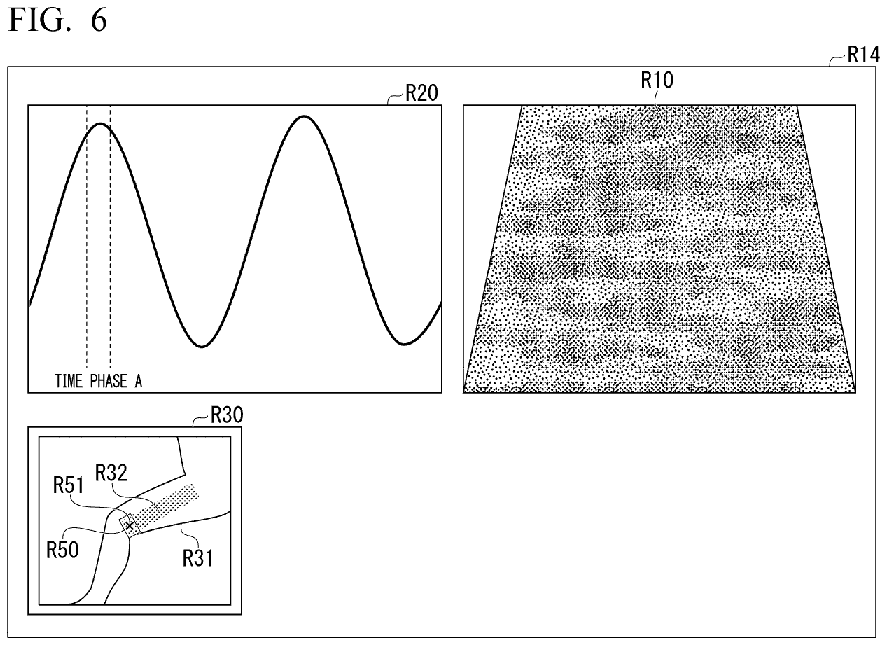

Next, an ultrasonic diagnostic apparatus 100 of a fourth embodiment will be described. FIG. 6 is a diagram showing an example of an image R14 displayed by the display 14 in the ultrasonic diagnostic apparatus 100 of the fourth embodiment. In FIG. 6, the acquisition range display image R30 displayed on the display 14 is represented and display of the ultrasonic image R10 and the time variation image R20 is omitted.

In the ultrasonic diagnostic apparatus 100 of the fourth embodiment, the acquisition function 122 outputs position information output from the magnetic sensor 30 to the display control function 134. The display control function 134 identifies a position of the ultrasonic probe 20 with respect to the body mark image R31 and a center position of the ultrasonic probe 20 on the basis of the position information output from the acquisition function 122.

The display control function 134 causes the display 14 to display a probe position image R50 representing the identified position of the ultrasonic probe 20 and a probe center position image R51 representing the center position of the ultrasonic probe 20 in addition to the body mark image R31 and the acquisition range image R32. The probe position image R50 may be displayed, for example, to surround a region having an area corresponding to the area of a transmission/reception face of the ultrasonic probe 20. The probe center position image R51 may be disposed, for example, at a centroid position in the probe position image R50 displayed on the display 14 and indicated by, for example, an X mark.

The ultrasonic diagnostic apparatus 100 of the fourth embodiment achieves the same effects as those of the ultrasonic diagnostic apparatus 100 of the first embodiment. In the ultrasonic diagnostic apparatus 100 of the fourth embodiment, the display control function 134 causes the display 14 to display the probe position image R50 representing the position of the ultrasonic probe 20 and the probe center position image R51 representing the center position thereof along with the body mark image R31 and the acquisition range image R32. Accordingly, an operator can easily recognize a positional relationship between a position of a subject at which an ultrasonic image cannot be captured and the ultrasonic probe 20. Therefore, it is possible to smoothly perform collection of ultrasonic images.

### Fifth Embodiment

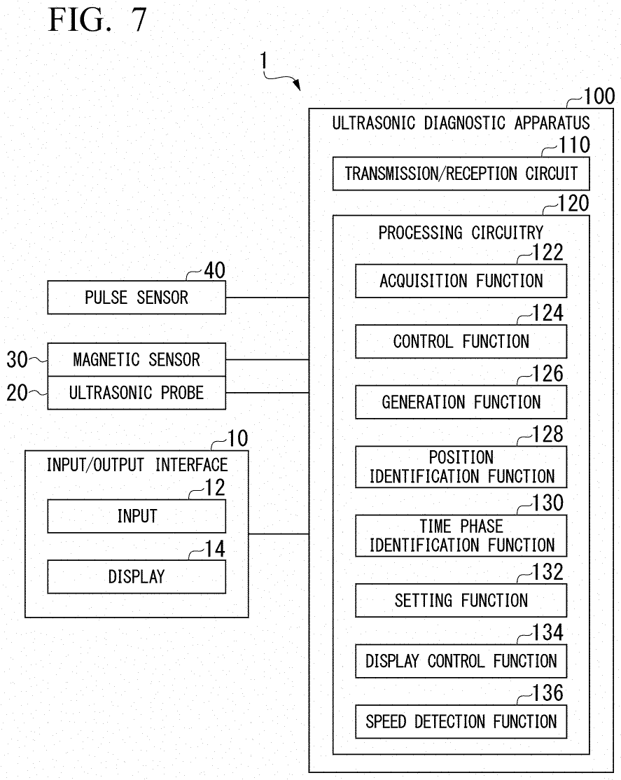

Next, an ultrasonic diagnostic apparatus 100 of a fifth embodiment will be described. FIG. 7 is a block diagram showing a functional configuration of an ultrasonic diagnostic system 1 of the fifth embodiment. In the ultrasonic diagnostic system 1 of the fifth embodiment, the processing circuitry 120 includes a speed detection function 136 in the ultrasonic diagnostic apparatus 100.

The speed detection function 136 marks a specific position of an ultrasonic image generated by the generation function 126 and detects a moving speed of the ultrasonic probe 20 on the basis of time variation in the marked specific position. In this case, the ultrasonic diagnostic apparatus 100 may mark the specific position, for example, by analyzing the ultrasonic image. A speed of the ultrasonic probe 20 is a feed speed of the ultrasonic probe 20. The speed detection function 136 is an example of a detector.

The speed detection function 136 outputs the detected moving speed of the ultrasonic probe 20 to the display control function 134. The display control function 134 identifies a moving speed of the ultrasonic probe 20 when an ultrasonic image has been acquired at a position in an acquisition range image (hereinafter referred to as a “moving speed at the time of capturing) on the basis of the speed of the ultrasonic probe 20 output from the speed detection function 136. The display control function 134 adjusts a display form of an acquisition range image R35 according to a moving speed at the time of capturing at each position in the acquisition range image.

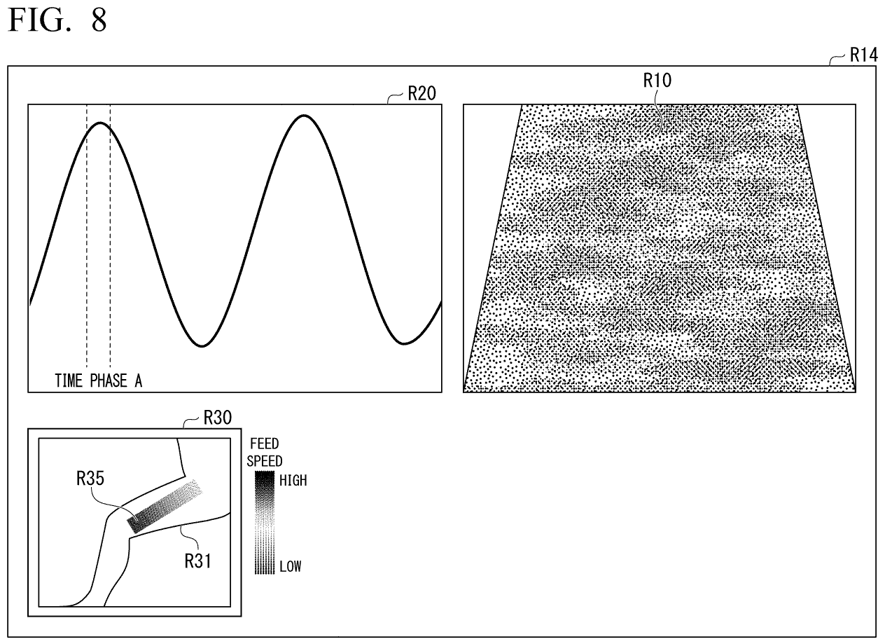

FIG. 8 is a diagram showing an example of an image R14 displayed by the display 14 in the ultrasonic diagnostic apparatus 100 of the fifth embodiment. The acquisition range display image R30 includes the body mark image R31 and the acquisition range image R35. The acquisition range image R35 may be, for example, an image in which visual display such as colors and shades of the acquisition range image varies according to display position.

In the acquisition range image R35, a moving speed at the time of capturing is high in a part displayed in dark, for example, in a first color, for example, red, and the moving speed at the time of capturing is low in a part displayed in dark, for example, in a second color, for example, blue, as shown in FIG. 8. The acquisition range image R35 is displayed in gradation such that the color becomes lighter as the moving speed at the time of capturing decreases in a state in which the moving speed at the time of capturing is high and the color becomes lighter as the moving speed at the time of capturing increases in a state in which the moving speed at the time of capturing is low. In FIG. 8, a moving speed at the time of capturing is high in a bottom left part in the acquisition range represented in the acquisition range image R35 and the moving speed at the time of capturing becomes lower towards the top right part. A moving speed at the time of capturing may be divided and displayed for respective speeds. A moving speed at the time of capturing may be displayed. in monochrome.

The ultrasonic diagnostic apparatus 100 of the fifth embodiment achieves the same effects as those of the ultrasonic diagnostic apparatus 100 of the first embodiment. The ultrasonic diagnostic apparatus 100 of the fifth embodiment adjust a display form of the acquisition range image R33 according to a moving speed at the time of capturing. Accordingly, an operator can easily ascertain an area in which the moving speed at the time of capturing is high and an area in which the moving speed at the time of capturing is low in an acquisition range image.

Although a moving speed of the ultrasonic probe 20 is detected through the speed detection function 136 in the fifth embodiment, a moving speed of the ultrasonic probe 20 may be detected through other detection means and the like. For example, a speed sensor provided in the ultrasonic probe 20 may be used, or a moving speed of the ultrasonic probe 20 may be detected by additionally providing a camera or the like which captures an optical image including the ultrasonic probe 20 and image-processing images captured by the camera.

### Sixth Embodiment

Next, an ultrasonic diagnostic apparatus 100 of a sixth embodiment will be described. The ultrasonic diagnostic apparatus 100 of the sixth embodiment detects a position of the ultrasonic probe 20, for example, on the basis of an ultrasonic image acquired by the acquisition function 122. In this case, the ultrasonic diagnostic apparatus 100 identifies a body part or the like of a subject, for example, by analyzing the ultrasonic image and detects a position of the ultrasonic probe 20 according to the position of the identified body part. Hereinafter, processing until an acquisition position and a time phase are labeled on an ultrasonic image in the ultrasonic diagnostic apparatus 100 of the sixth embodiment will be described.

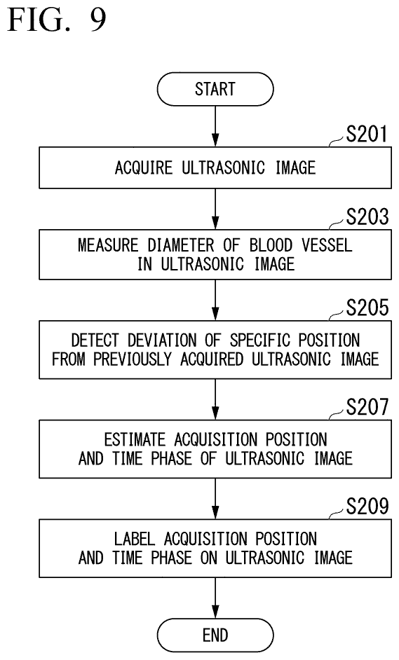

FIG. 9 is a flowchart showing an example of processing of the ultrasonic diagnostic apparatus 100 of the sixth embodiment. The ultrasonic diagnostic apparatus 100 may execute processing of the flowchart shown in FIG. 9, for example, whenever the ultrasonic probe 20 outputs reflected wave information to the ultrasonic diagnostic apparatus 100. First, the ultrasonic diagnostic apparatus 100 of the sixth embodiment generates an ultrasonic image through the generation function 126 on the basis of reflected wave information output from the ultrasonic probe 20 (step S201) and outputs the ultrasonic image to the acquisition function 122. The acquisition function 122 stores the acquired ultrasonic image in a storage device. The storage device stores ultrasonic images acquired by the acquisition function 122 after the ultrasonic diagnostic apparatus 100 initiates acquisition of ultrasonic images.

Subsequently, the generation function 126 analyzes the ultrasonic image to search for a blood vessel in the ultrasonic image and measures the diameter of the searched blood vessel (step S203). Subsequently, the generation function 126 detects a deviation of a specific position in the ultrasonic image on the basis of changes in the position and diameter of the blood vessel through comparison between the ultrasonic image and an ultrasonic image previously acquired by the generation function 126 (step S205). Subsequently, the position identification function 128 estimates an acquisition position of the ultrasonic image and the time phase identification function 130 estimates a time phase in which the ultrasonic probe 20 has received a reflected echo signal for generating the ultrasonic image on the basis of the deviation of the specific position detected by the generation function 126 (step S207).

Thereafter, the ultrasonic diagnostic apparatus 100 labels the acquisition position estimated by the position identification function 128 and the time phase estimated by the time phase identification function 130 on the ultrasonic image acquired by the acquisition function 122 through the display control function 134 (step S209). In this manner, the ultrasonic diagnostic apparatus 100 ends processing shown in FIG. 9.

The ultrasonic diagnostic apparatus 100 of the sixth embodiment estimates, on the basis of an ultrasonic image generated from reflected wave information output from the ultrasonic probe 20, an acquisition position and a time phase of the ultrasonic image and labels the acquisition position and the time phase on the ultrasonic image. Accordingly, it is not necessary to provide the magnetic sensor 30 and the like for detecting an acquisition position, and thus the apparatus can be simplified.

### Seventh Embodiment

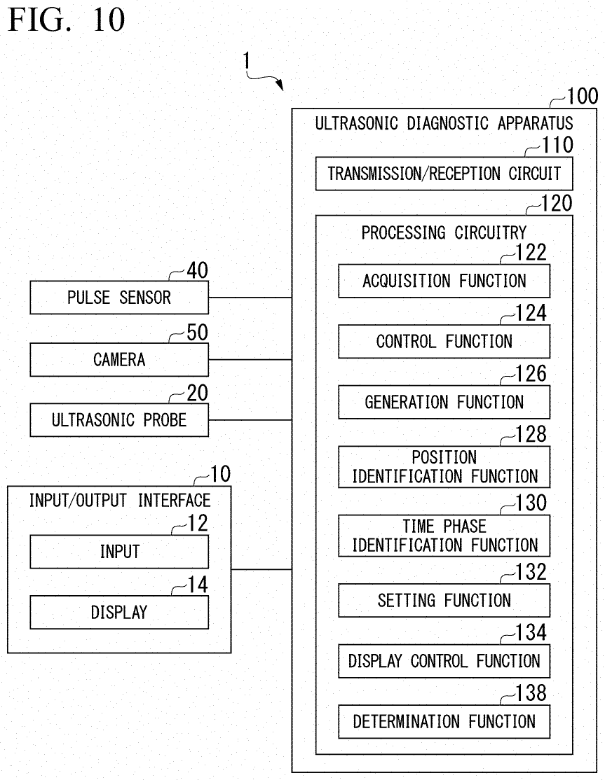

Next, an ultrasonic diagnostic apparatus 100 of a seventh embodiment will be described. FIG. 10 is a block diagram showing a functional configuration of an ultrasonic diagnostic system 1 of the seventh embodiment. In the ultrasonic diagnostic apparatus 100 of the seventh embodiment, the generation function 126 stores acquired ultrasonic images in a storage device to collect them as in the ultrasonic diagnostic apparatus 100 of the sixth embodiment. The ultrasonic diagnostic apparatus 100 of the seventh embodiment may create a composite image, for example, on the basis of a plurality of collected ultrasonic images.

In creation of a composite image, it is necessary to satisfy creation conditions for creating the composite image. The ultrasonic diagnostic apparatus 100 of the seventh embodiment includes a determination function 138. The ultrasonic diagnostic apparatus 100 stores the creation conditions in the storage device, and the determination function 138 determines whether the creation conditions for creating a composite image are satisfied according to a plurality of ultrasonic images acquired and collected by the acquisition function 122. The determination function 138 determines the creation conditions for each specific time phase area. The determination function 138 is an example of a determiner.

The determination function 138 determines whether all ultrasonic images at acquisition positions necessary to create a composite image have been collected on the basis of a plurality of ultrasonic images generated by the generation function 126 and acquired and collected by the acquisition function 122. When the determination function determines that the creation conditions are not satisfied, the display control function 134 identifies an acquisition range deficient to satisfy the creation conditions (hereinafter referred to as a “deficient range”). The display control function 134 visualizes the deficient range and causes the display 14 to display the visualized deficient range.

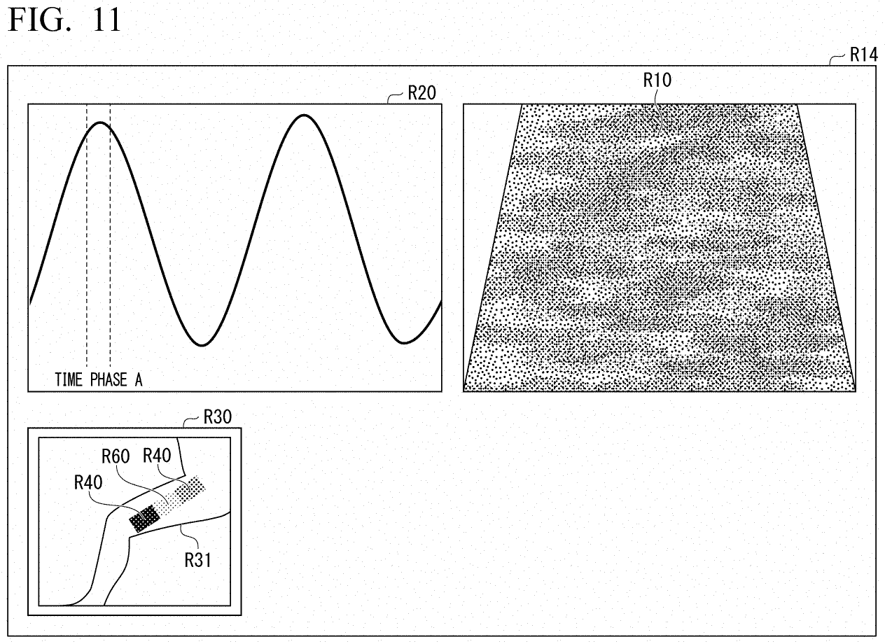

FIG. 11 is a diagram showing an example of an image R14 displayed by the display 14 of the seventh embodiment. The display control function 134 displays a deficient range image R60 along with acquisition range images R40. The deficient range image R60 is displayed to be clearly distinguished from the acquisition range images R40. The deficient range image R60 may be displayed in gradation between the acquisition range images R40.

For example, when a composite image of a blood vessel is attempted to be created, ultrasonic images including one end, the middle part, and the other end of the blood vessel are required. Here, it is assumed that the acquisition function 122 has acquired ultrasonic images of one end and the other end of the blood vessel but has not acquired an ultrasonic image of the middle part of the blood vessel. In this case, the display control function 134 causes the display 14 to display the acquisition range images R40 and the deficient range image such that the acquisition range images R40 are superimposed on parts of one end and the other end of the blood vessel and the deficient range image is superimposed on the middle part. When there is an area in which ultrasonic images can be collected with a necessary density for creation of the composite image, the display control function 134 may extend a collection range to the area and display the collection range.

The ultrasonic diagnostic apparatus 100 of the seventh embodiment causes the display 14 to display an acquisition range for acquiring ultrasonic images necessary to create a composite image through the display control function 134. Accordingly, it is possible to easily acquire ultrasonic images necessary to create a composite image. Furthermore, it is possible to notify an ultrasonic image collection state and to prevent incomplete collection of ultrasonic images.

### Eighth Embodiment

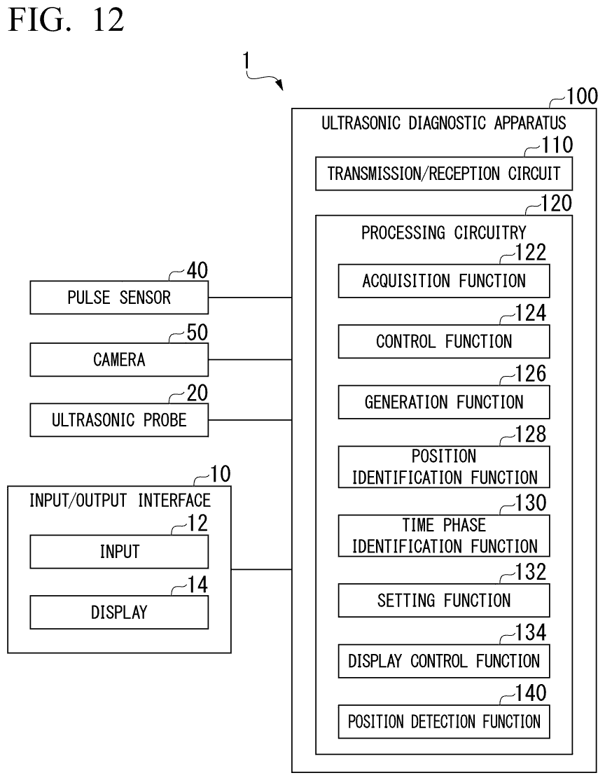

Next, an ultrasonic diagnostic apparatus 100 of an eighth embodiment will be described. FIG. 12 is a block diagram showing a functional configuration of an ultrasonic diagnostic system 1 of the eighth embodiment. The ultrasonic diagnostic system 1 of the eighth embodiment includes a camera 50 that images the ultrasonic probe 20 and does not include the magnetic sensor 30 shown in FIG. 1. The ultrasonic diagnostic apparatus 100 includes a position detection function 140. The camera 50 is an example of an imager.

The camera 50 captures an optical image including the ultrasonic probe 20. The camera 50 outputs the captured optical image to the ultrasonic diagnostic apparatus 100. The position detection function 140 in the ultrasonic diagnostic apparatus 100 detects a position of the ultrasonic probe 20 by performing image processing on the optical image output from the camera 50. The position detection function 140 outputs the acquired position of the ultrasonic probe 20 to the position identification function 128.

The ultrasonic diagnostic apparatus 100 of the eighth embodiment detects a position of the ultrasonic probe 20 on the basis of the optical image output from the camera 50 that images the ultrasonic probe 20. Accordingly, it is not necessary to provide the magnetic sensor 30 and the like in the ultrasonic probe 20, and thus it is possible to suppress an increase in the weight of the ultrasonic probe 20 and to easily handle the ultrasonic probe 20.

According to at least one of the above-described embodiments, it is possible to confirm a spatiotemporal collection state of images by including an acquirer which acquires an image of a subject based on a reflected echo signal obtained from reflection of an ultrasonic signal, which is transmitted from an ultrasonic probe, from the subject, a position identifier which identifies an acquisition position in the subject at which the image has been acquired, a time phase identifier which identifies a time phase in which the image has been acquired, and a display controller which causes a display to display an acquisition range of the image on the subject in at least one specific time phase area based on the identified acquisition position and time phase.

Although several embodiments have been described, these embodiments have been suggested as examples and are not intended to limit the scope of the invention. These embodiments can be implemented in other various forms and various omissions, substitutions and modifications are possible without departing from the essential characteristics of the invention. These embodiments and modifications thereof are included in the scope and essential characteristics of the invention and also included in the invention disclosed in claims and the equivalents thereof.

What is claimed is:

1. An ultrasonic diagnostic apparatus, comprising processing circuitry configured to:
acquire an image of a subject based on a reflected echo signal obtained from reflection of an ultrasonic signal, which is transmitted from an ultrasonic probe, from the subject;
identify an acquisition position in the subject at which the image has been acquired;
identify a time phase in which the image has been acquired; and
cause a display to display an acquisition range of the image on the subject in at least one specific time phase area based on the identified acquisition position and time phase.

2. The ultrasonic diagnostic apparatus according to claim 1, wherein
the processing circuitry is configured to identify the acquisition position based on a detection result of a sensor provided in the ultrasonic probe.

3. The ultrasonic diagnostic apparatus according to claim 1, wherein
the processing circuitry is configured to identify the acquisition position based on the acquired image.

4. The ultrasonic diagnostic apparatus according to claim 1, further comprising an imager configured to image the ultrasonic probe,
wherein the processing circuitry is configured to identify the acquisition position based on the image imaged by the imager.

5. The ultrasonic diagnostic apparatus according to claim 1, wherein
the processing circuitry is configured to identify the time phase based on biometric information about a heart rate or a pulse of the subject.

6. The ultrasonic diagnostic apparatus according to claim 1, wherein
the processing circuitry is configured to identify the time phase based on information about a cardiac cycle of the subject.

7. The ultrasonic diagnostic apparatus according to claim 1, wherein
the processing circuitry is configured to set the specific time phase area.

8. The ultrasonic diagnostic apparatus according to claim 1, wherein
the processing circuitry is configured to cause the display to display a body mark, the acquisition range being caused to be visually displayed on the body mark.

9. The ultrasonic diagnostic apparatus according to claim 1, wherein
the processing circuitry is configured to cause the display to display a reference image, the acquisition range being caused to be visually displayed on the reference image.

10. The ultrasonic diagnostic apparatus according to claim 1, wherein
the processing circuitry is configured to cause the display to display a plurality of acquisition ranges having different specific time phase areas side by side.

11. The ultrasonic diagnostic apparatus according to claim 1, wherein
the processing circuitry is configured to cause a plurality of acquisition ranges having different specific time phase areas from one another to be overlapped and displayed on the display.

12. The ultrasonic diagnostic apparatus according to claim 1, wherein
the processing circuitry is configured to adjust a display form of the acquisition range according to a collection rate of the images.

13. The ultrasonic diagnostic apparatus according to claim 1, wherein
the processing circuitry is configured to:
identify a position of the ultrasonic probe; and
cause the identified position of the ultrasonic probe and the acquisition range to be overlapped and displayed on the display.

14. The ultrasonic diagnostic apparatus according to claim 1, wherein
the processing circuitry is configured to:
detect a moving speed of the ultrasonic probe at the time of acquiring the image; and
adjust a display form of the acquisition range according to the detected moving speed.

15. The ultrasonic diagnostic apparatus according to claim 1, wherein
the processing circuitry is configured to:
determine whether creation conditions for creating a composite image using a acquired and collected plurality of images are satisfied; and
cause the display to display the acquisition range necessary to satisfy the creation conditions when determining that the creation conditions are not satisfied.

16. A non-transitory computer-readable storage medium storing a program, the program causing an ultrasonic diagnostic apparatus to:
acquire an image of a subject based on a reflected echo signal obtained from reflection of an ultrasonic signal, which is transmitted from an ultrasonic probe, from the subject;
identify an acquisition position in the subject at which the image has been acquired;
identify a time phase in which the image has been acquired; and
cause a display to display an acquisition range of the image on the subject in at least one specific time phase area based on the identified acquisition position and time phase.
